(19) United States
(12) Patent Application Publication
(10) Pub. No.: US 2025/0262083 A1
(43) Pub. Date: Aug. 21, 2025

(54) PELVIC BINDING APPARATUS, SYSTEMS, AND METHODS

(71) Applicant: Alphapointe, Kansas City, MO (US)

(72) Inventors: CHRISTOPHER B. KOSIOREK, La Vernia, TX (US); Esra Abir, New York, NY (US); Brenda Mee, West Newfield, ME (US); Ryan Williams, Olathe, KS (US)

(21) Appl. No.: 19/030,448

(22) Filed: Jan. 17, 2025

Related U.S. Application Data
(63) Continuation of application No. 17/589,830, filed on Jan. 31, 2022.
Application No. 17/589,830 is a continuation-in-part of application No. 16/258,225, filed on Jan. 25, 2019, now Pat. No. 11,234,854.
(60) Provisional application No. 62/621,949, filed on Jan. 25, 2018.

(51) Int. Cl. A61F 5/058

(52) U.S. Cl. CPC A61F 5/058

(57) ABSTRACT

A foldable pelvic binding device and method of use is provided. The device includes a main body divided into a plurality of panels and segments by a grid of living hinges, thereby facilitating movement between stowed and flat configurations. The main body is configured to couple with one or more other main body, as necessary or desired, to create a binding device that is sized for the required application, thereby accommodating a variety of applications in a variety of situations for a variety of body shapes and sizes. By engaging the binding device with a tourniquet, a binding system is created. When not in use, each the main body of the binding device can be moved to the stowable configuration by folding the various panels over each other.

### CROSS-REFERENCE TO RELATED APPLICATIONS

This application is a continuation application of U.S. patent application Ser. No. 17/589,830, filed Jan. 31, 2022, which is a continuation-in-part application of U.S. patent application Ser. No. 16/258,225, filed Jan. 25, 2019, now U.S. Pat. No. 11,234,854, which claims priority pursuant to 35 U.S.C. 119(e) to U.S. Provisional Patent Application Ser. No. 62/621,949, filed Jan. 25, 2018, the entire disclosures of which are incorporated herein by reference.

### FIELD

The present invention relates generally to medical devices and related systems and methods. More specifically, the present invention is concerned with a rigid immobilization system that is foldable for ease of storage and use.

### BACKGROUND

Existing splints, pelvic binders, and other immobilization devices (“splints”) are either too cumbersome and/or heavy when not in use or too flimsy when in use. Furthermore, existing splints do not provide the versatility required in the field, often requiring users to haul a variety of unnecessary pieces and/or components, increasing the risk that one or more such piece or component will be missing when needed. Furthermore still, many splint/binding devices are designed for one-time use and/or can only be used feasibly a few times or less. Consequently, it would be beneficial to have a splint/binding system that is lightweight and durable. Furthermore, it would be beneficial if the splint were capable of being easily moved between a stowed configuration for ease of storage and a variety of deployed configurations for use. Furthermore still, it would be beneficial if the splint were formed from a material and with a configuration so as to facilitate multiple uses and to minimize loss of any necessary pieces or components.

### SUMMARY

The present inventive concept is an immobilization device. It includes an elongated main body and two or more panels with a living hinge between each panel. Each panel also includes two or more segments with a living hinge between each segment. The main body is moveable between multiple configurations. In the flat configuration, the panels and segments are all unfolded and flat relative to one another. In the stowable configuration, the panels are folded over one another to minimize the surface area of the main body. The main body may also be moved to various deployed configurations. In each deployed configuration, the angle between two adjacent panels is at or between 0 and 180 degrees. In each deployed configuration, the outermost segments of each panel are folded at an angle between 0 and 180 degrees with respect to the next adjacent segment of that panel. Folding along the hinges between segments provides strength and rigidity to the device in the lengthwise direction when the device is in the various deployed configurations.

In some embodiments, the main body includes one or more hole or slot, sized and shaped such that they align when the main body is folded into the stowable configuration. The holes may also be sized and shaped to receive a strap, band, cord, cable, nail, pin, or other fastener. The fastener through the holes secures the device in the stowable configuration or the deployed configuration. In some embodiments, the immobilization device is configured to be used with a tourniquet such that compressive loads are distributed across a larger area than is possible through the use of a tourniquet alone, such as a pelvic binder. Sometimes, the device is used with a tourniquet where the tourniquet is the fastener securing the device in place.

The device in each deployed configuration is configured to restrain an appendage, limb, or extremity in various different predetermined positions, such as 180 degree orientation (straight arm or leg), 45 degrees, 90 degrees, 135 degrees, etc. The main body includes one or more pinch point configured to establish and maintain these various different predetermined angles.

The foregoing and other objects are intended to be illustrative of the invention and are not meant in a limiting sense. Many possible embodiments of the invention may be made and will be readily evident upon a study of the following specification and accompanying drawings comprising a part thereof. Various features and subcombinations of invention may be employed without reference to other features and subcombinations. Other objects and advantages of this invention will become apparent from the following description taken in connection with the accompanying drawings, wherein is set forth by way of illustration and example, an embodiment of this invention and various features thereof.

### BRIEF DESCRIPTION OF THE DRAWINGS

A preferred embodiment of the present inventive concept, illustrative of the best mode in which the applicant has contemplated applying the principles, is set forth in the following description and is shown in the drawings and is particularly and distinctly pointed out and set forth in the appended claims.

FIG. 1 is a perspective view of a first embodiment of an immobilization device of the present invention, the main body shown in a flat configuration, broken lines showing of holes and slots through the main body are for the purpose of showing additional optional embodiments.

FIG. 2 is a front view of the embodiment shown in FIG. 1.

FIG. 3 is a rear view of the embodiment shown in FIG. 1.

FIG. 4 is a first end view of the embodiment shown in FIG. 1.

FIG. 5 is a second end view of the embodiment shown in FIG. 1.

FIG. 6 is a first side view of the embodiment shown in FIG. 1.

FIG. 7 is a second side view of the embodiment shown in FIG. 1.

FIG. 8 is a perspective view of a second embodiment of an immobilization device of the present invention, the main body shown in a flat configuration, broken lines showing of large and small slots through the main body are for the purpose of showing additional optional embodiments.

FIG. 9 is a front view of the embodiment shown in FIG. 8.

FIG. 10 is a rear view of the embodiment shown in FIG. 8.

FIG. 11 is a first end view of the embodiment shown in FIG. 8.

FIG. 12 is a second end view of the embodiment shown in FIG. 8.

FIG. 13 is a first side view of the embodiment shown in FIG. 8.

FIG. 14 is a second side view of the embodiment shown in FIG. 8.

FIG. 15 is a perspective view of the embodiment shown in FIG. 8, the main body shown in a first deployed configuration with a first portion of the main body being generally perpendicular to a second portion of the main body.

FIG. 16 is a front view of the embodiment shown in FIG. 15.

FIG. 17 is a rear view of the embodiment shown in FIG. 15.

FIG. 18 is a first side view of the embodiment shown in FIG. 15.

FIG. 19 is a second side view of the embodiment shown in FIG. 15.

FIG. 20 is a top view of the embodiment shown in FIG. 15.

FIG. 21 is a bottom view of the embodiment shown in FIG. 15.

FIG. 22 is a perspective view of the embodiment shown in FIG. 8, the main body shown in a second deployed configuration with a first portion of the main body being angled away from a second portion of the main body.

FIG. 23 is a front view of the embodiment shown in FIG. 22.

FIG. 24 is a rear view of the embodiment shown in FIG. 22.

FIG. 25 is a first side view of the embodiment shown in FIG. 22.

FIG. 26 is a second side view of the embodiment shown in FIG. 22.

FIG. 27 is a top view of the embodiment shown in FIG. 22.

FIG. 28 is a bottom view of the embodiment shown in FIG. 22.

FIG. 29 is a perspective view of the embodiment shown in FIG. 8, the main body shown in a third deployed configuration with a first portion of the main body being angled away from a second portion of the main body.

FIG. 30 is a front view of the embodiment shown in 29.

FIG. 31 is a rear view of the embodiment shown in 29.

FIG. 32 is a first side view of the embodiment shown in 29.

FIG. 33 is a second side view of the embodiment shown in 29.

FIG. 34 is a top view of the embodiment shown in 29.

FIG. 35 is a bottom view of the embodiment shown in 29.

FIG. 36 is a perspective view of a third embodiment of an immobilization device of the present invention, the main body shown in a flat configuration, broken lines showing of holes and slots through the main body are for the purpose of showing additional optional embodiments.

FIG. 37 is a front view of the embodiment shown in FIG. 36.

FIG. 38 is a rear view of the embodiment shown in FIG. 36.

FIG. 39 is a first end view of the embodiment shown in FIG. 36.

FIG. 40 is a second end view of the embodiment shown in FIG. 36.

FIG. 41 is a first side view of the embodiment shown in FIG. 36.

FIG. 42 is a second side view of the embodiment shown in FIG. 36.

FIG. 43 is a perspective view of the embodiment shown in FIG. 36, the main body shown in a first deployed configuration with a first portion of the main body being generally perpendicular to a second portion of the main body.

FIG. 44 is a front view of the embodiment shown in FIG. 43.

FIG. 45 is a rear view of the embodiment shown in FIG. 43.

FIG. 46 is a first side view of the embodiment shown in FIG. 43.

FIG. 47 is a second side view of the embodiment shown in FIG. 43.

FIG. 48 is a top view of the embodiment shown in FIG. 43.

FIG. 49 is a bottom view of the embodiment shown in FIG. 43.

FIG. 50 is a perspective view of the embodiment shown in FIG. 36, the main body shown in a second deployed configuration with a first portion of the main body being angled away from a second portion of the main body.

FIG. 51 is a front view of the embodiment shown in FIG. 50.

FIG. 52 is a rear view of the embodiment shown in FIG. 50.

FIG. 53 is a first side view of the embodiment shown in FIG. 50.

FIG. 54 is a second side view of the embodiment shown in FIG. 50.

FIG. 55 is a top view of the embodiment shown in FIG. 50.

FIG. 56 is a bottom view of the embodiment shown in FIG. 50.

FIG. 57 is a front view of a fourth embodiment of an immobilization device of the present invention, the main body shown in a stowable configuration.

FIG. 58 is a front view of a fifth embodiment of an immobilization device of the present invention, the main body shown in a stowable configuration.

FIG. 59 is a front view of a sixth embodiment of an immobilization device of the present invention, the main body shown in a stowable configuration.

FIG. 60 is a cross-sectional view of a seventh embodiment of an immobilization device of the present invention, the main body shown in a deployed configuration used in connection with a tourniquet as a pelvic binder.

FIG. 61 is an image of a main body assembly of an embodiment of the present invention, the main body assembly having a plurality of snap components coupled to a main body, the main body defining a plurality of engagement slots.

FIG. 61 is a 2artial view of the main body assembly of FIG. 61 shown on an enlarged scale, the main body including instructions for moving the main body assembly to an angled configuration and for securing the main body assembly in such configuration.

FIG. 63 is an image of instructions printed on the main body assembly of FIG. 61, the instructions depicting a method of moving the main body assembly to an angled configuration and securing the main body assembly to a limb of a user when in such configuration.

FIG. 64 is an image of instructions printed on the main body assembly of FIG. 61, the instructions depicting a method of moving the main body to a straight configuration and securing the main body assembly to a limb of a user when in such configuration.

FIG. 65 is an image of instructions printed on the main body assembly of FIG. 61, the instructions depicting a method of utilizing the main body with a tourniquet for pelvic splinting.

### DETAILED DESCRIPTION

The following detailed description of the invention references the accompanying drawings that illustrate specific embodiments in which the invention can be practiced. The embodiments are intended to describe aspects of the invention in sufficient detail to enable those skilled in the art to practice the invention. Other embodiments can be utilized and changes can be made without departing from the scope of the present invention. The following detailed description is, therefore, not to be taken in a limiting sense.

In this description, references to “one embodiment,” “an embodiment,” or “embodiments” mean that the feature or features being referred to are included in at least one embodiment of the technology. Separate references to “one embodiment,” “an embodiment,” or “embodiments” in this description do not necessarily refer to the same embodiment and are also not mutually exclusive unless so stated and/or except as will be readily apparent to those skilled in the art from the description. For example, a feature, structure, act, etc. described in one embodiment may also be included in other embodiments, but is not necessarily included. Thus, the present technology can include a variety of combinations and/or integrations of the embodiments described herein.

In some embodiments, the present inventive concept is an immobilization device 10. The immobilization device is used to splint or immobilize a bodily extremity. The immobilization device includes an elongated main body 101. The main body includes a plurality of panels 102 with a hinged panel connection 103 between each panel and its adjacent panel(s). Each panel includes a plurality of segments 104 with a hinged segment connection 105 between each segment and its adjacent segment(s). In some embodiments, a panel connection angle is defined by the geometric angle between two adjacent panels. In some embodiments, a segment connection angle is defined by the geometric angle between two adjacent segments. In some embodiments, the hinged panel connection is a living hinge. In some embodiments, the hinged segment connection is a living hinge. In some embodiments, the entire main body is formed of a single piece of material and the hinged panel connection(s) and the hinged segment connection(s) are living hinges. In some embodiments, the main body is formed from metal, polypropylene, polyethylene, or other plastics.

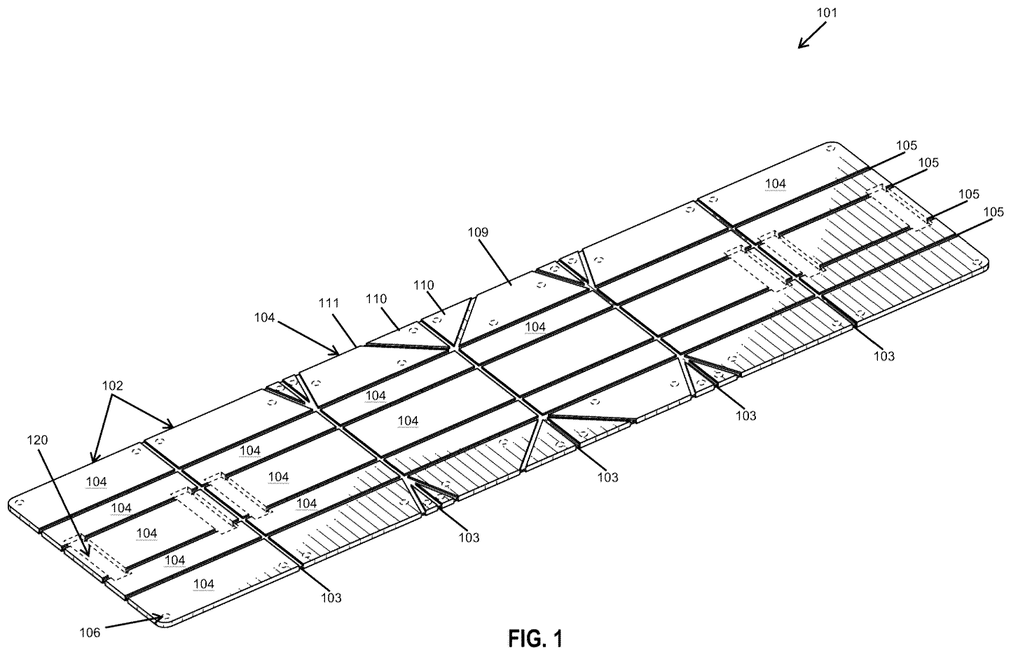

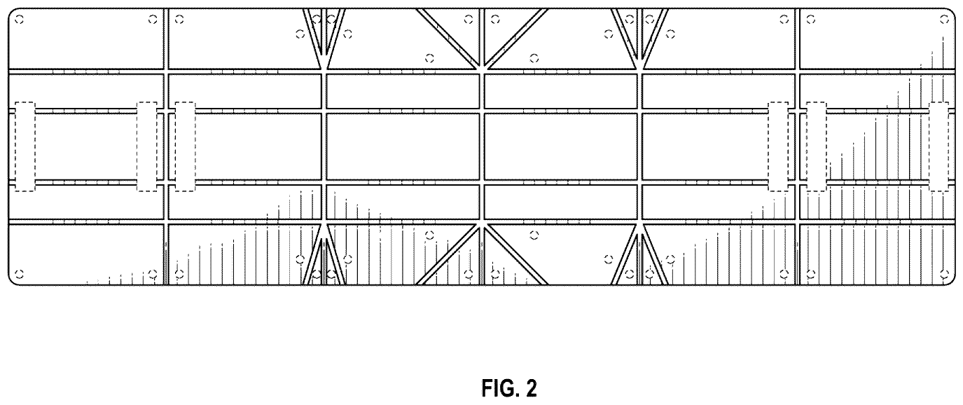

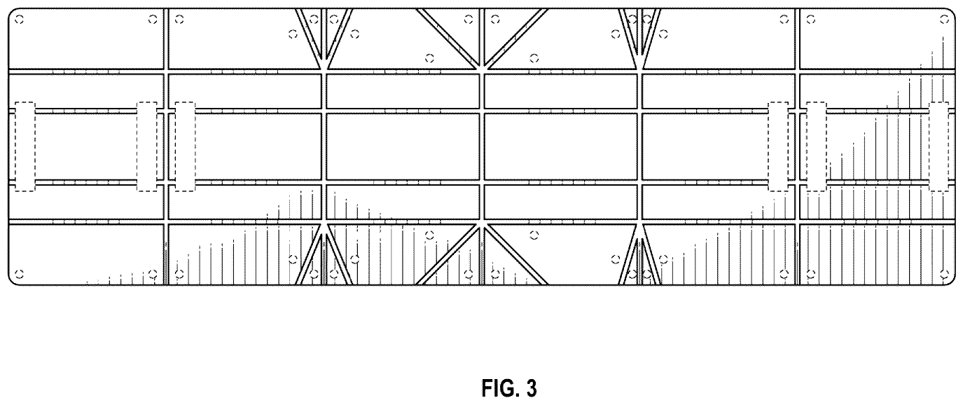

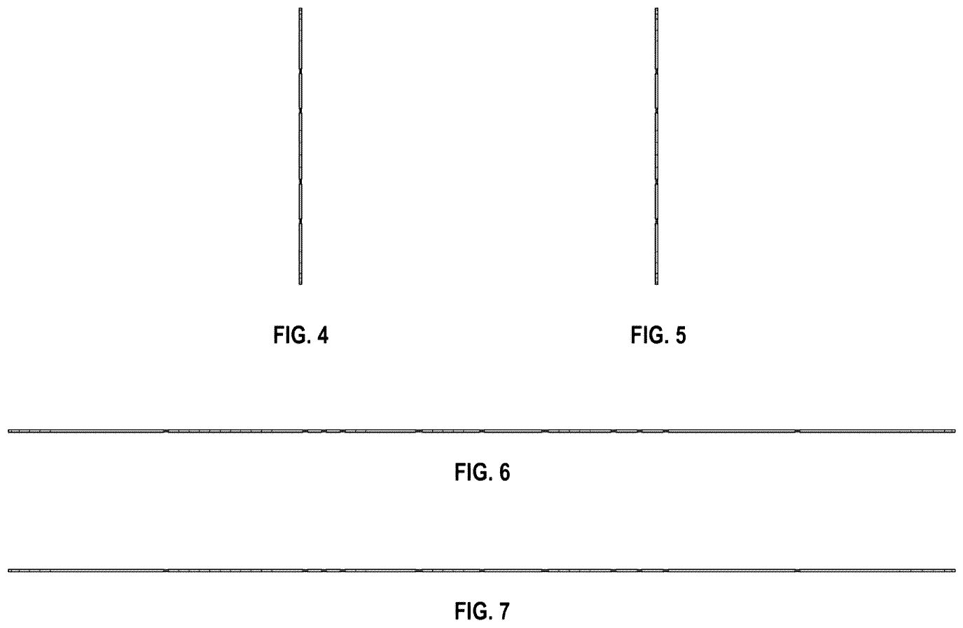

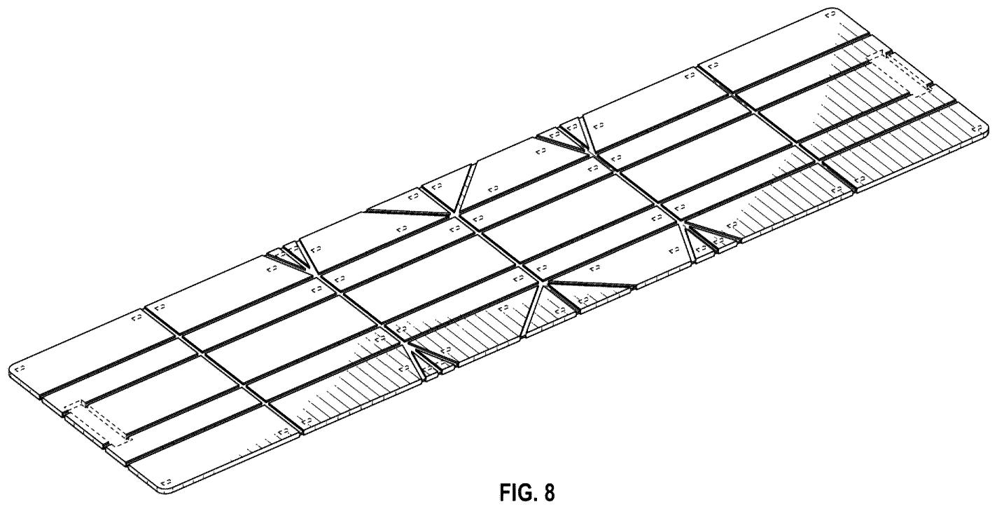

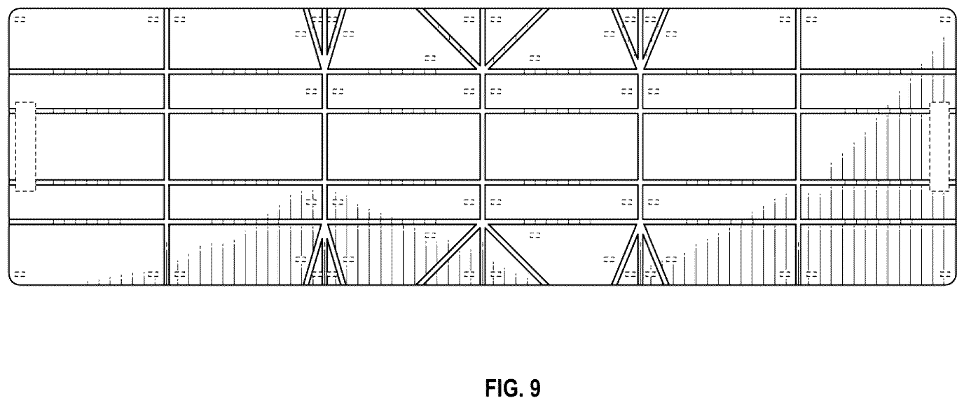

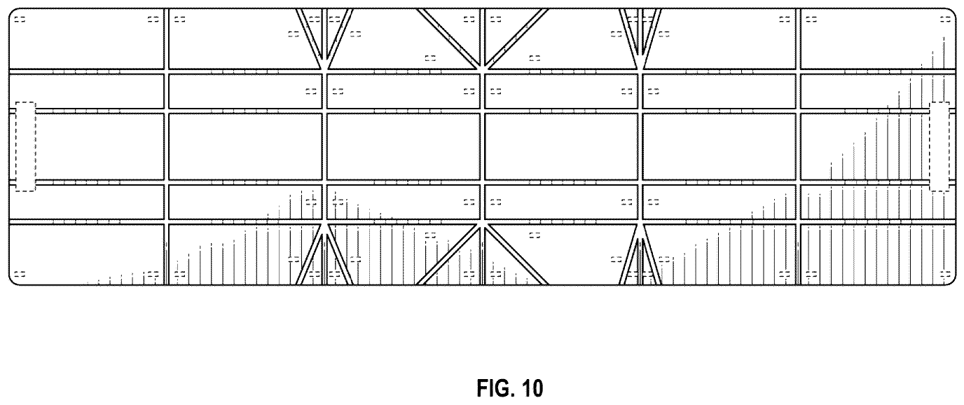

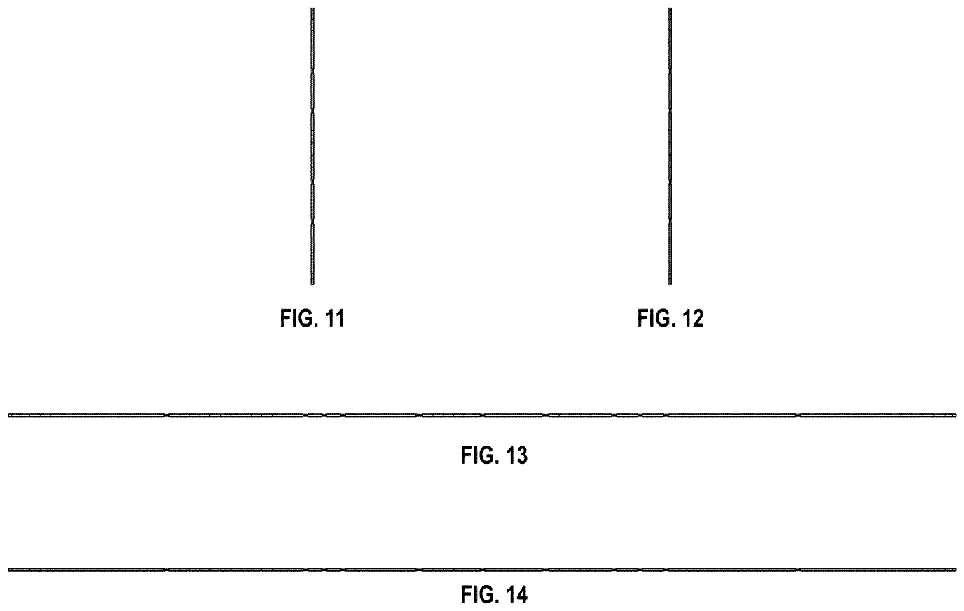

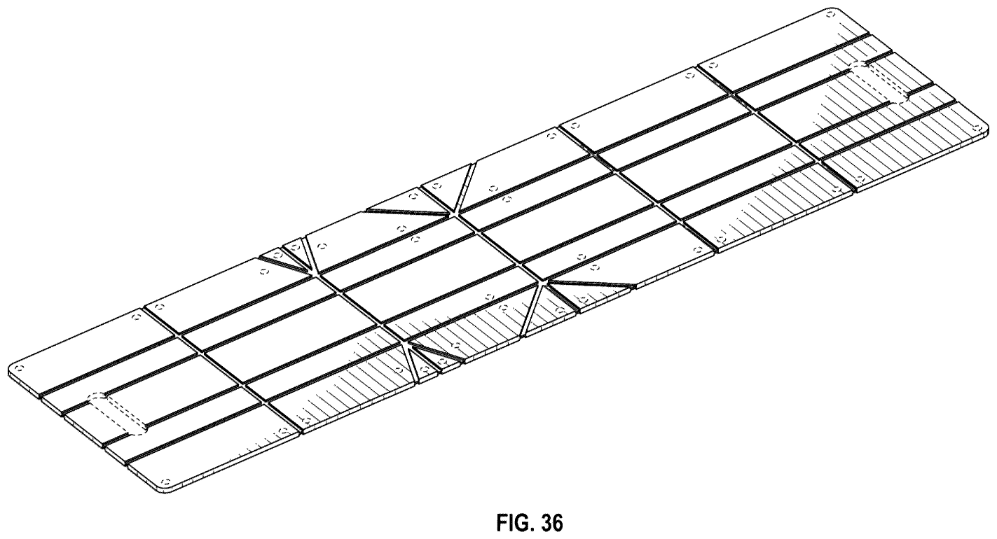

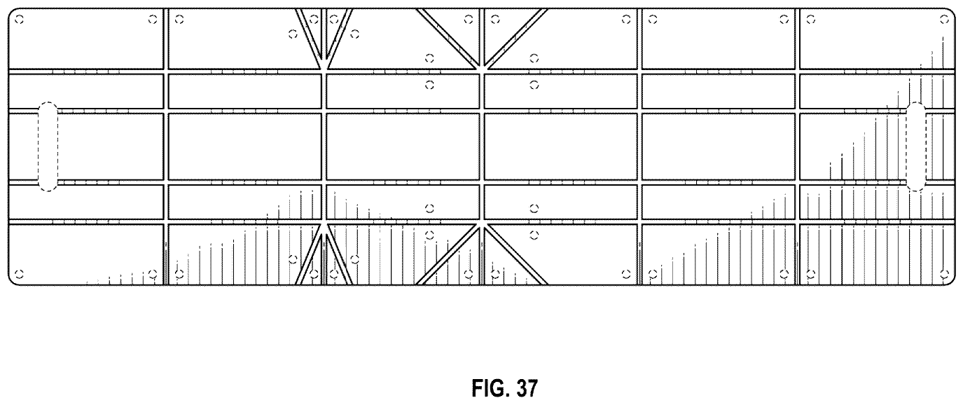

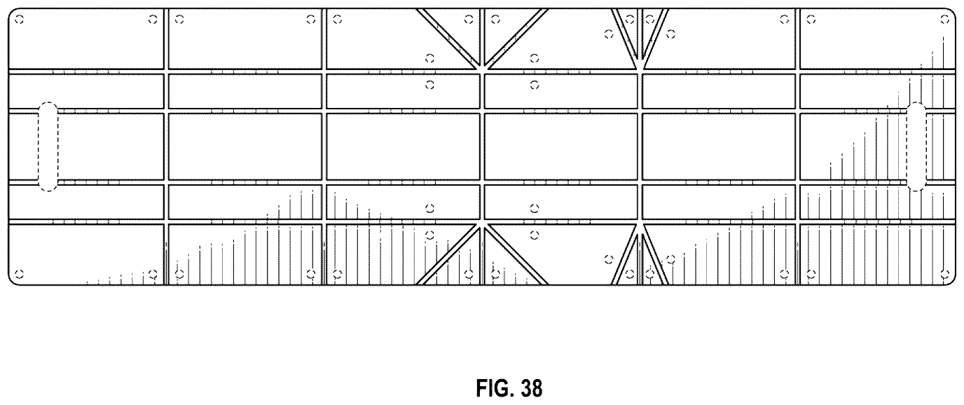

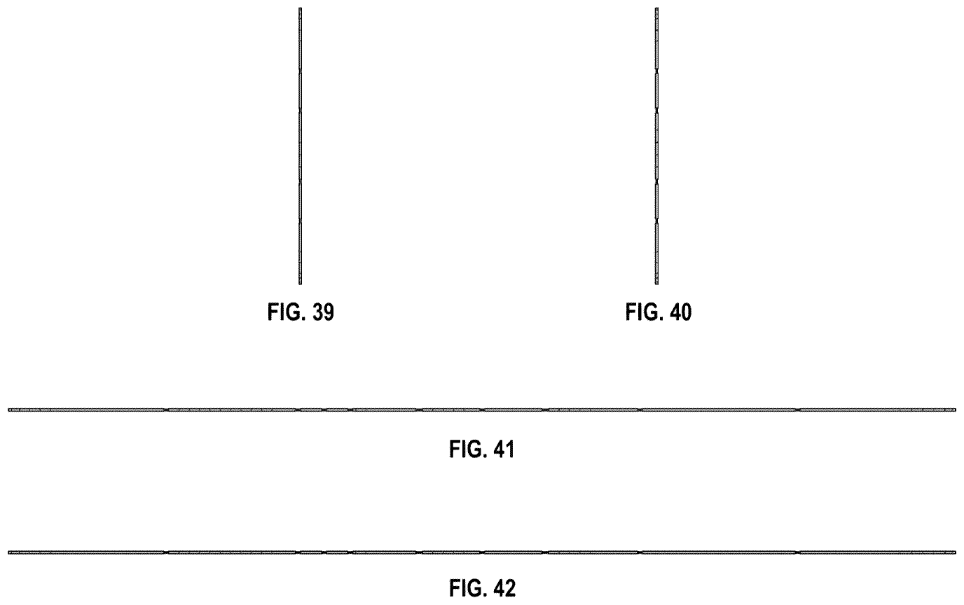

Referring to FIGS. 1-14 and 36-42, in some embodiments, the main body is unfolded into a flat configuration. Each panel is unfolded and flat with respect to its adjacent panel(s). In the flat configuration, the panel connection angle is 180 degrees. In other words, the panels are all aligned and parallel in the flat configuration. In the flat configuration, the segment connection angle is 180 degrees. In other words, the segments within each panel remain flat with respect to one another and also with respect to the segments of adjacent panels.

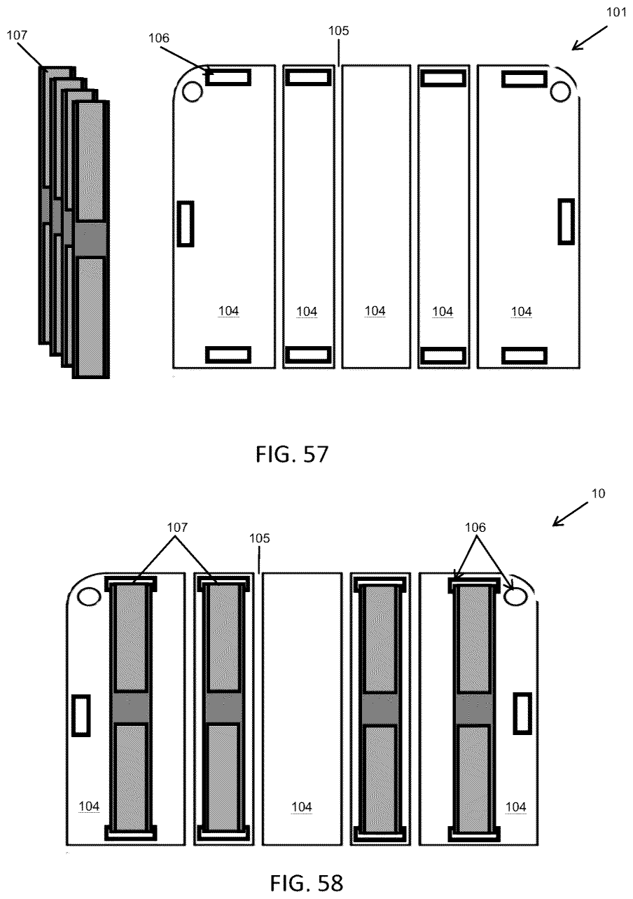

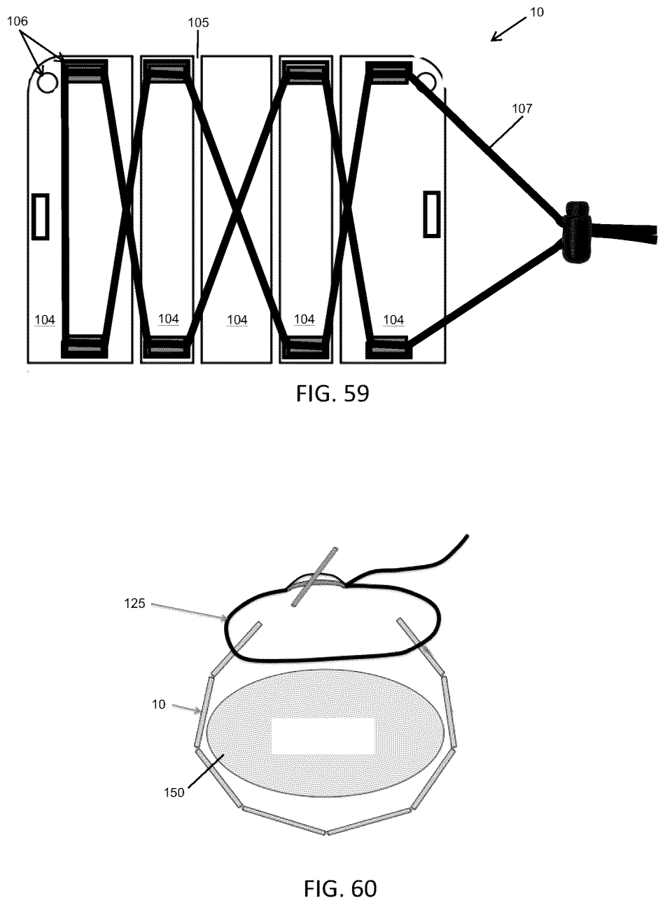

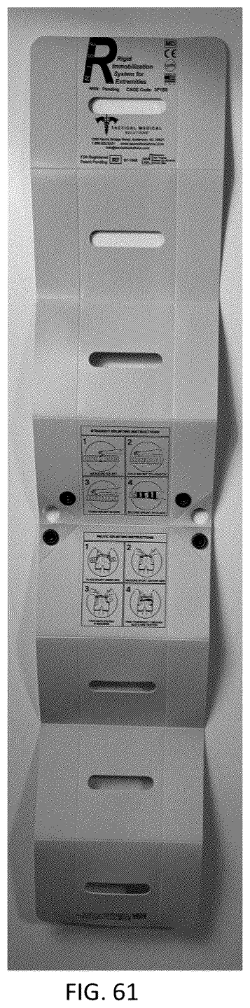

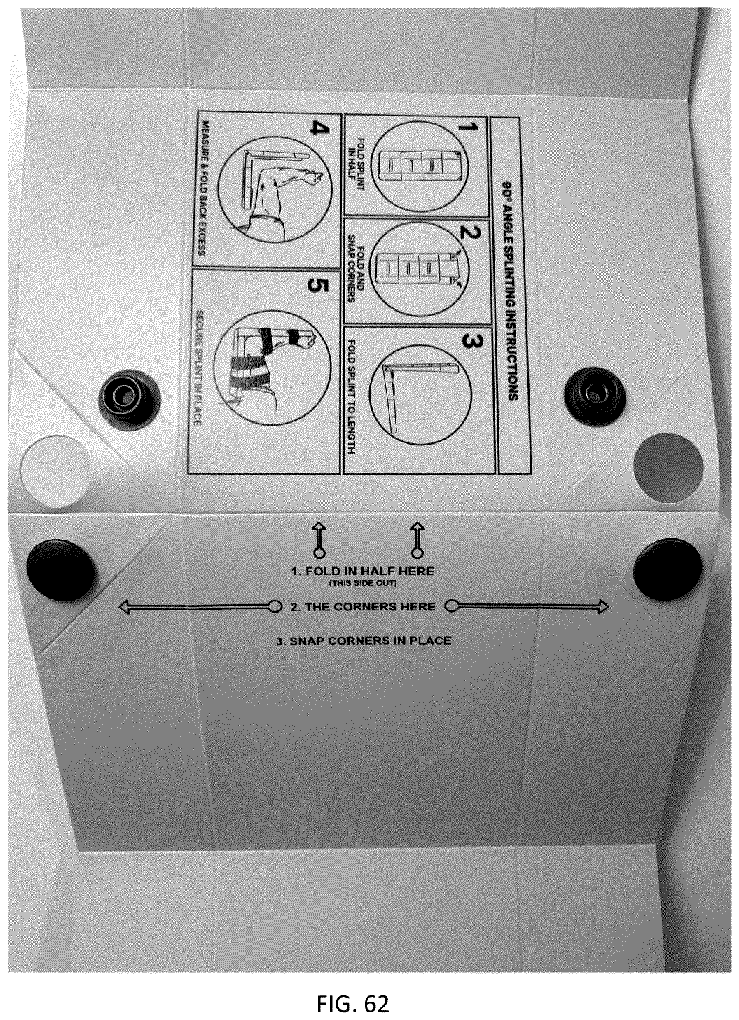

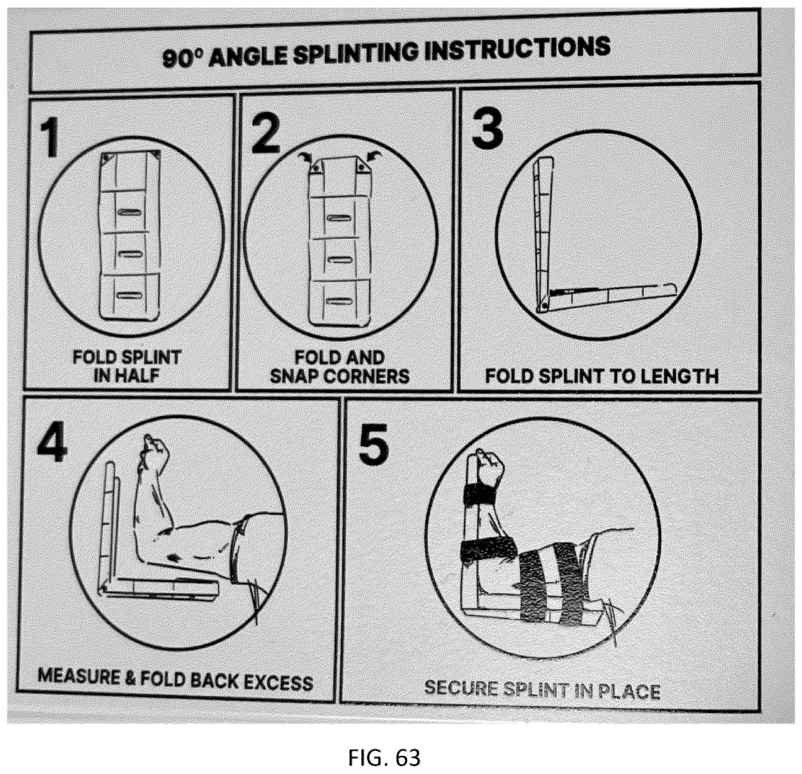

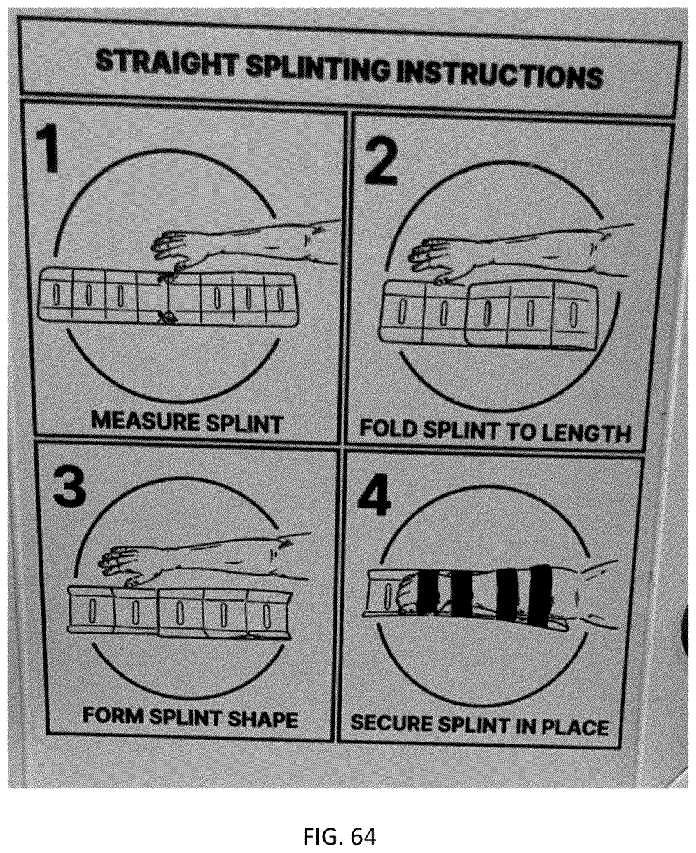

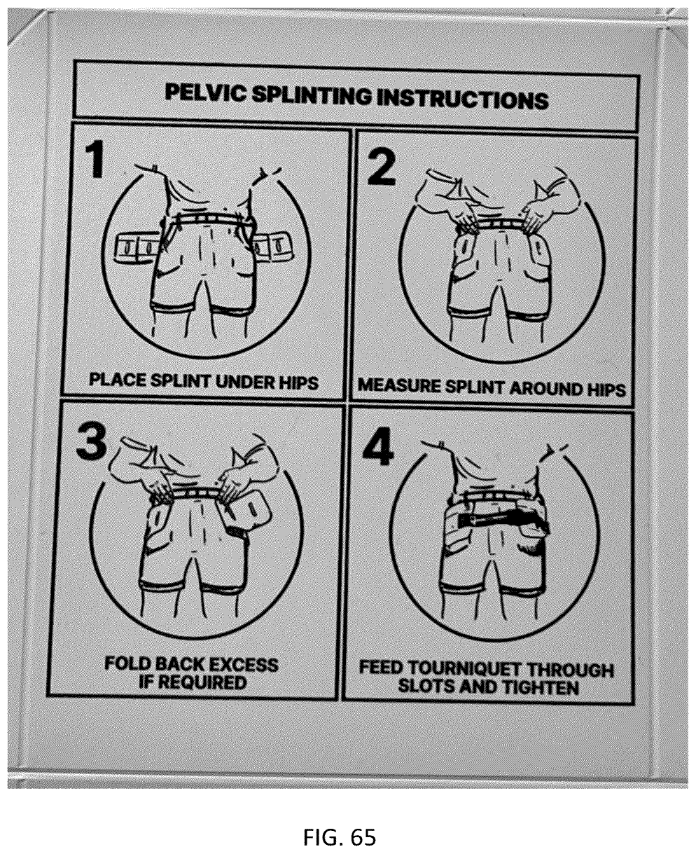

Referring to FIGS. 57, 58, and 59, in some embodiments, the main body is folded into a stowable configuration. Each panel is folded over adjacent panel(s) to minimize the surface area of the main body. In the stowable configuration, the panel connection angle is zero degrees. In other words, the panels fold on top of one another, accordion-style. In the stowable configuration, the segment connection angle is 180 degrees. In other words, the segments within each panel remain flat with respect to one another. Within each panel, the segments are not folded when the main body is in the stowable configuration.

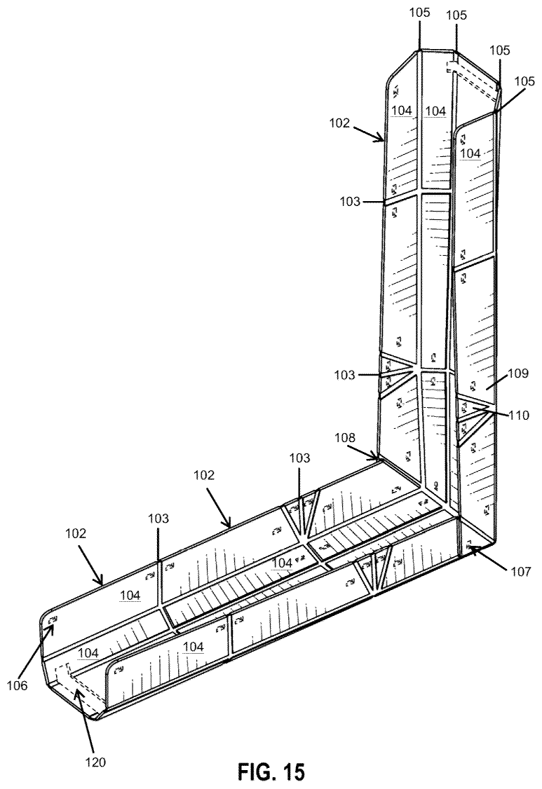

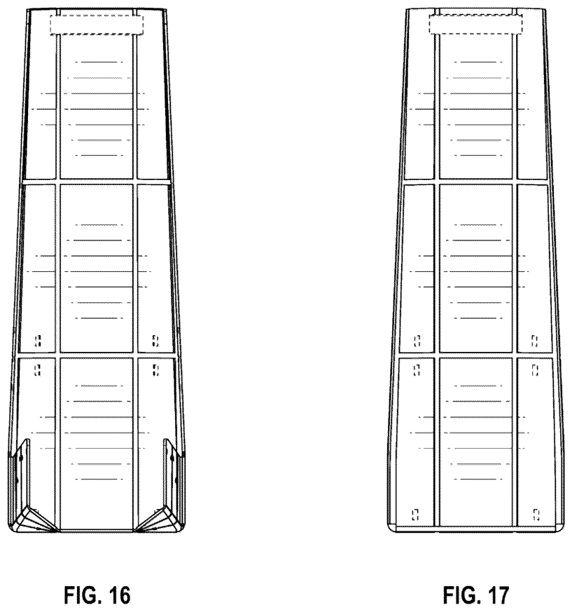

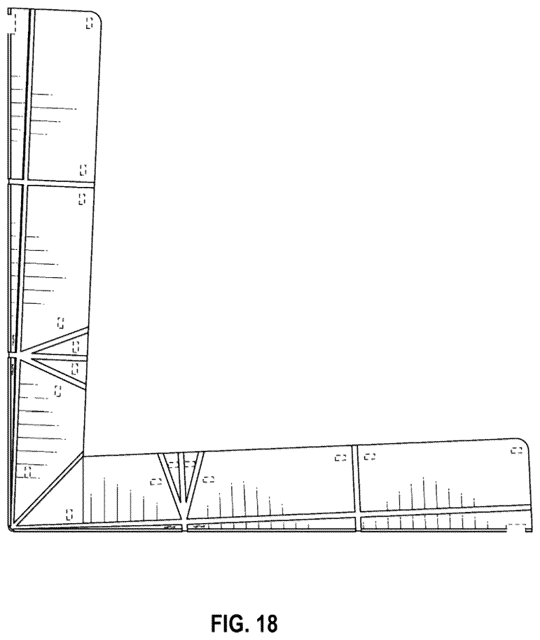

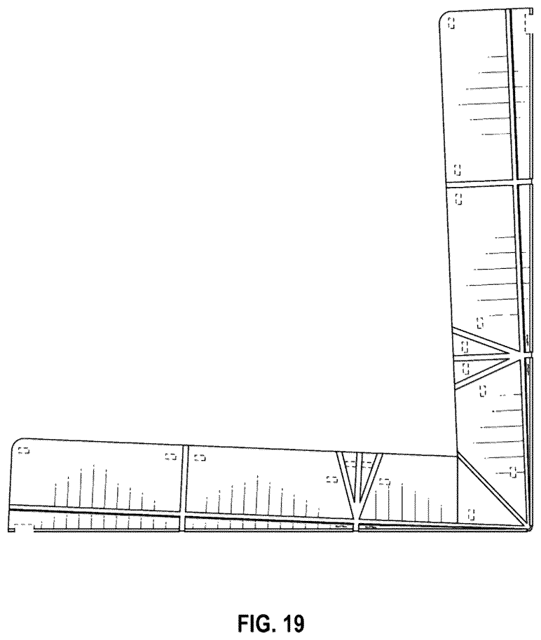

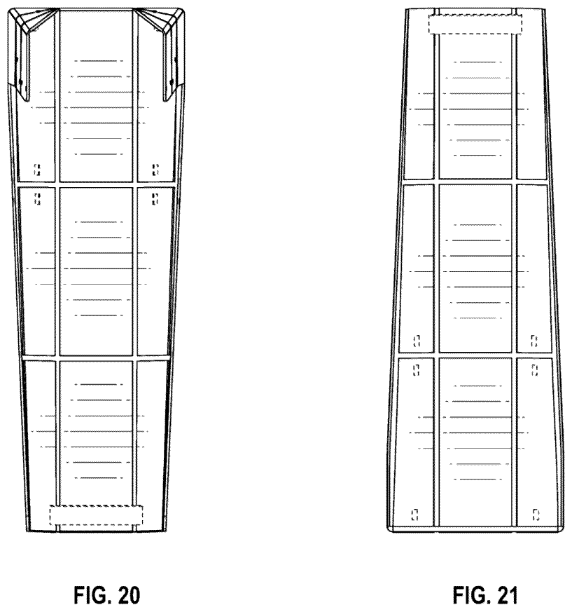

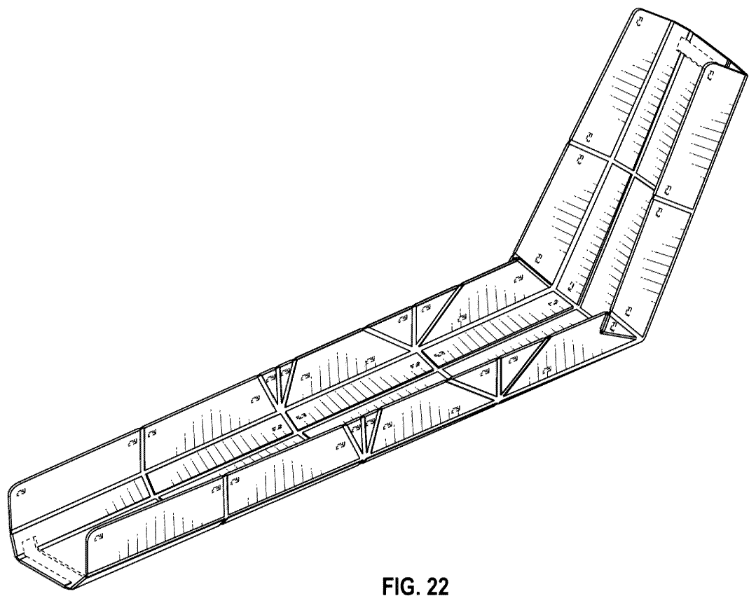

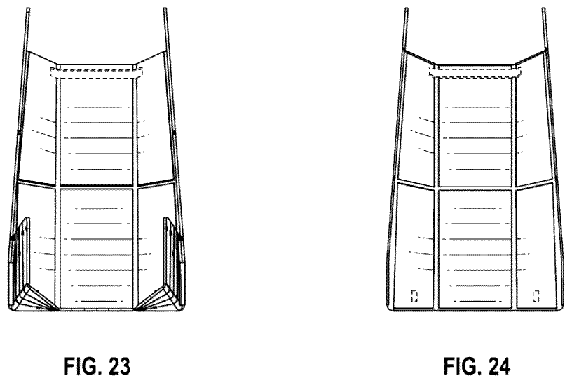

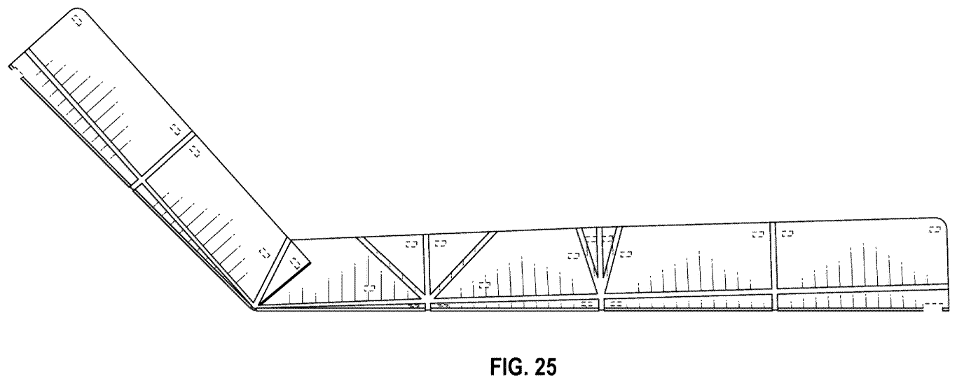

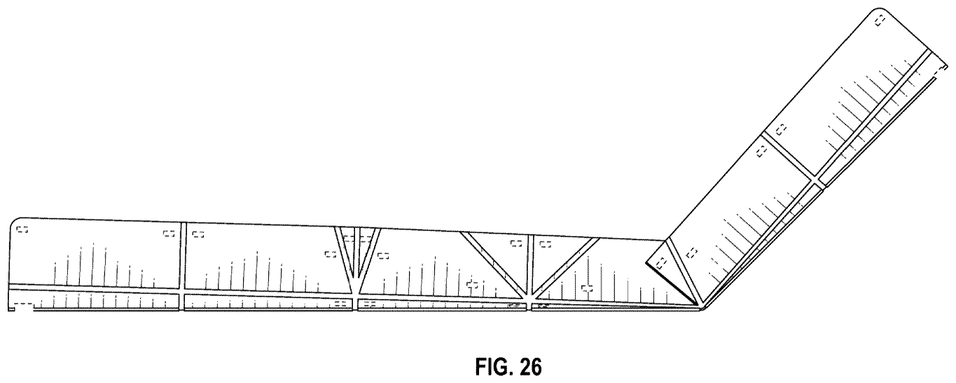

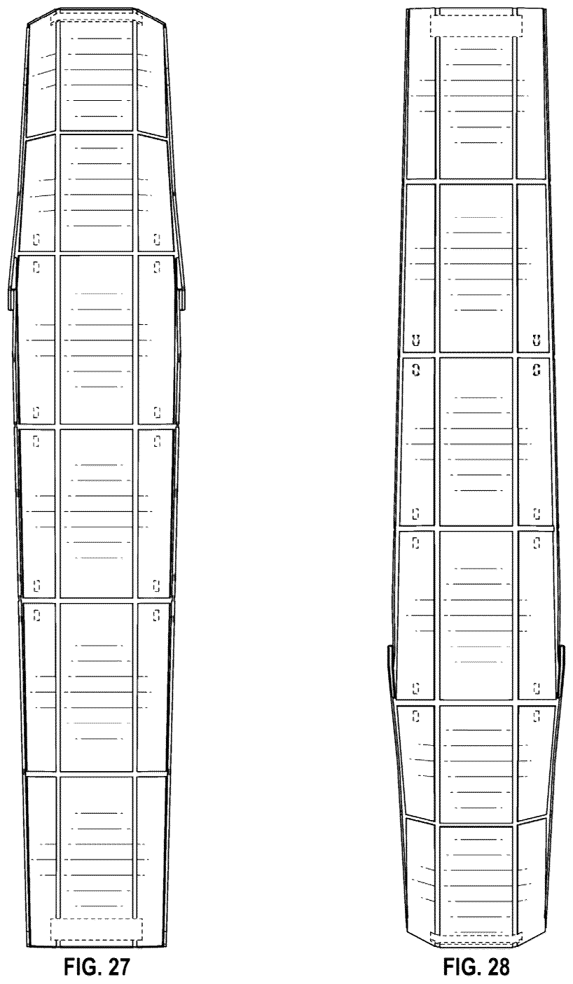

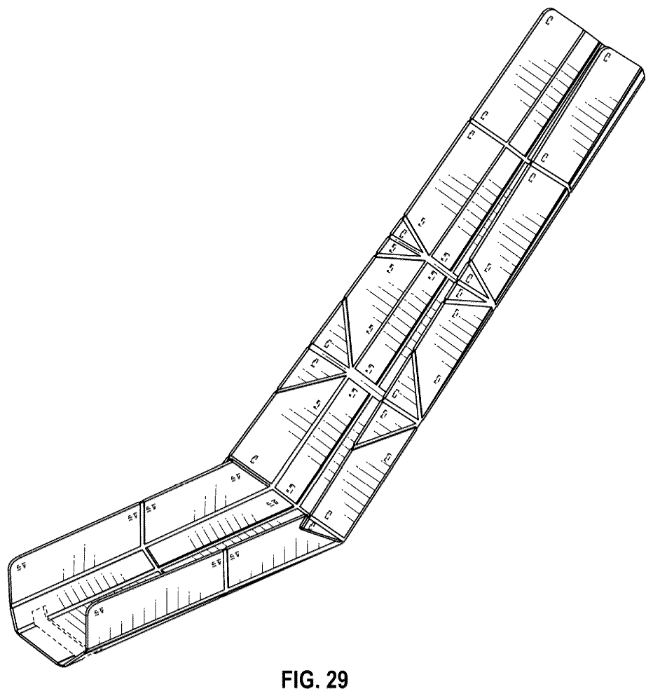

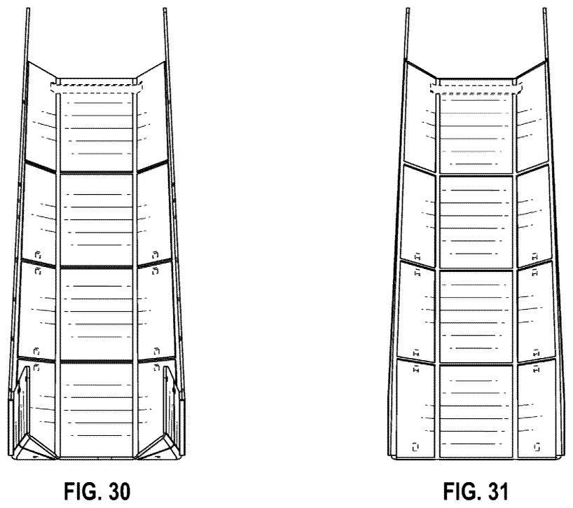

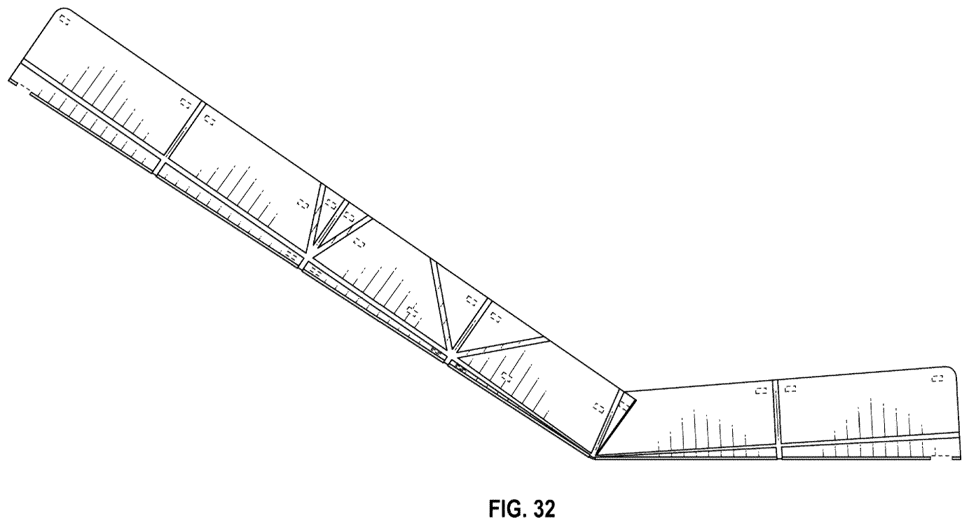

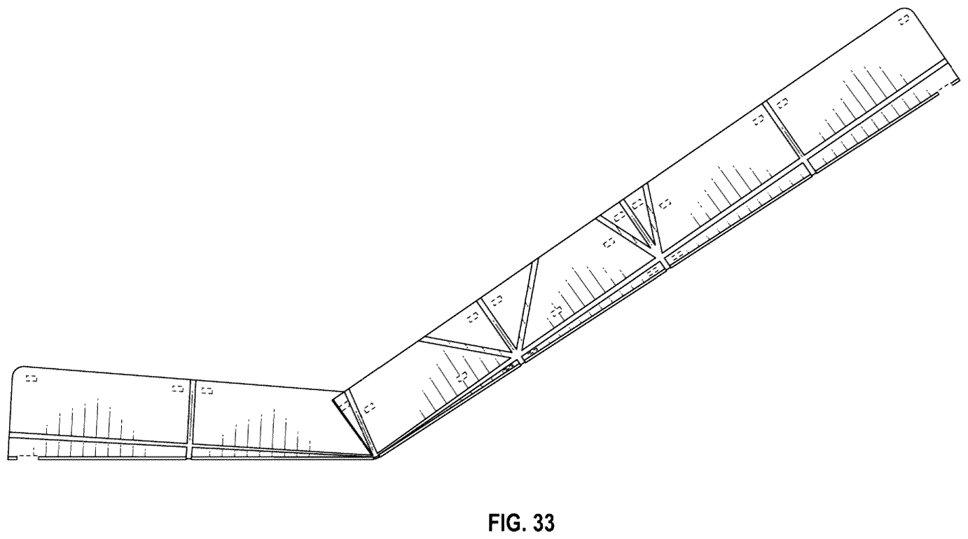

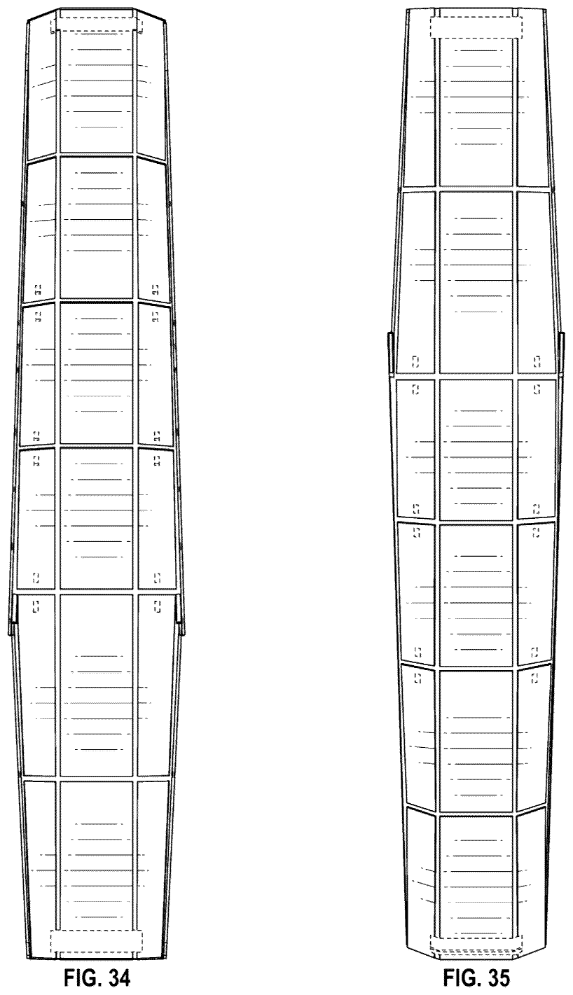

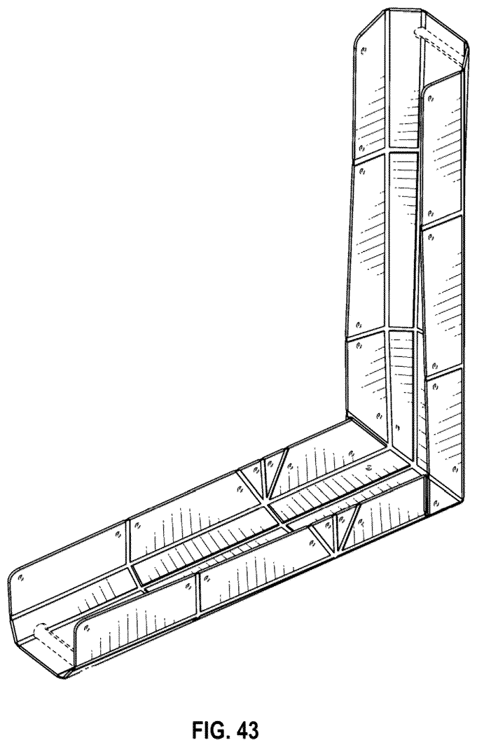

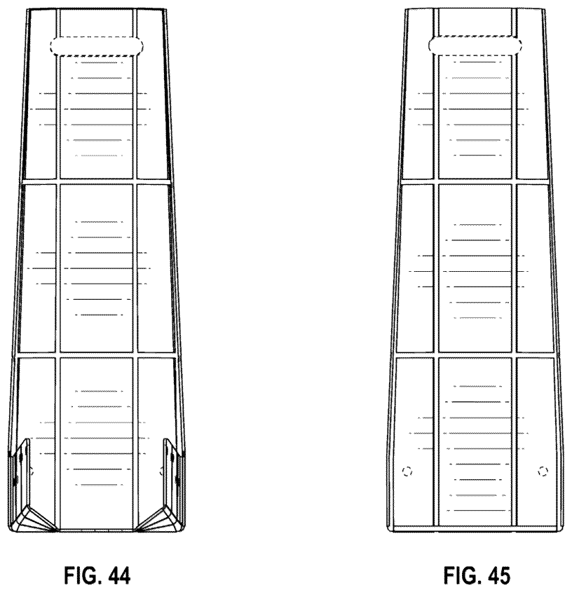

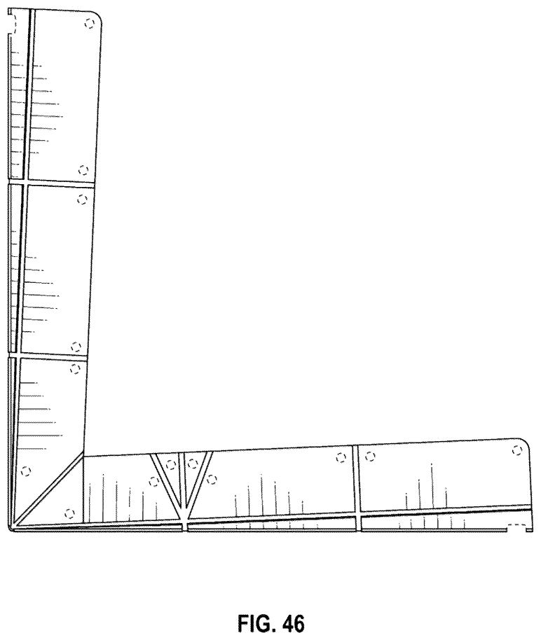

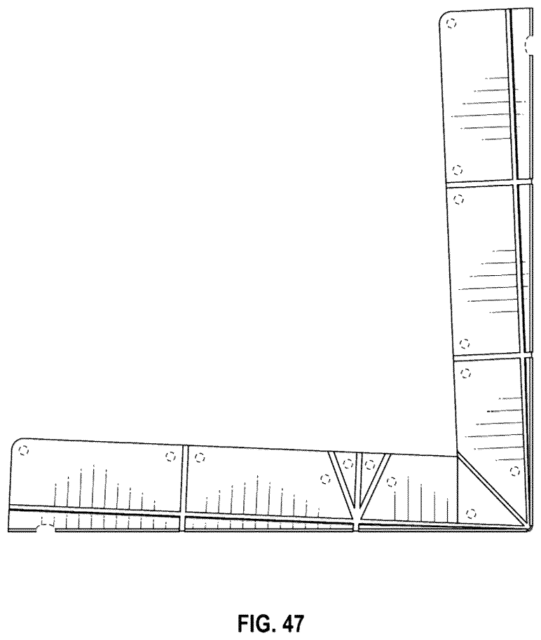

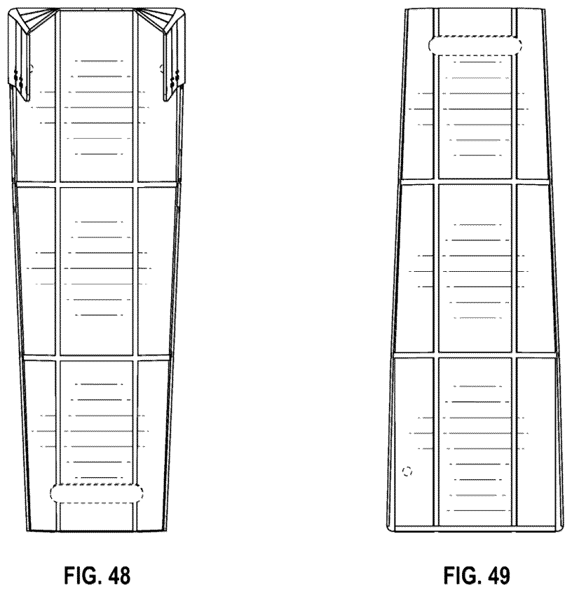

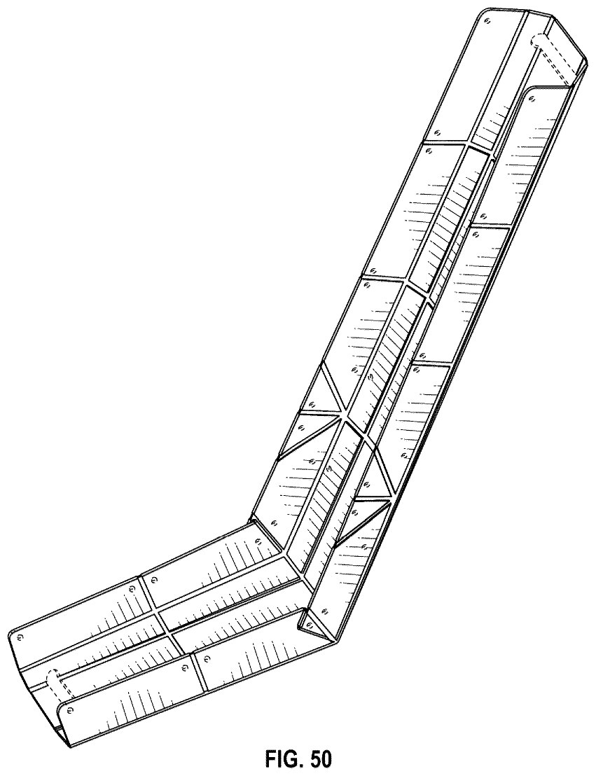

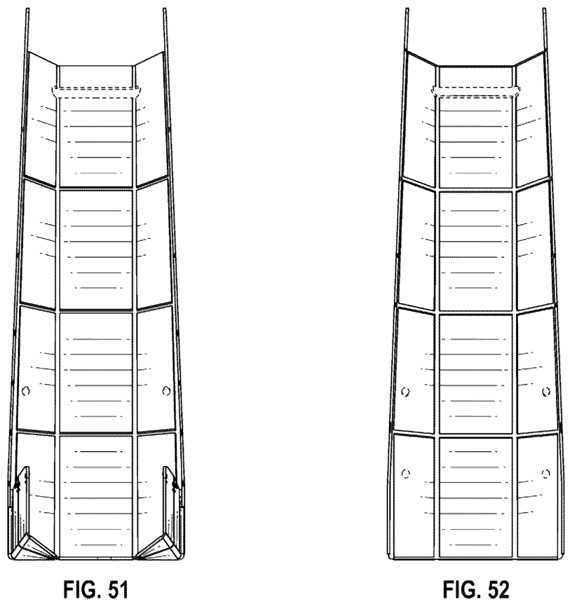

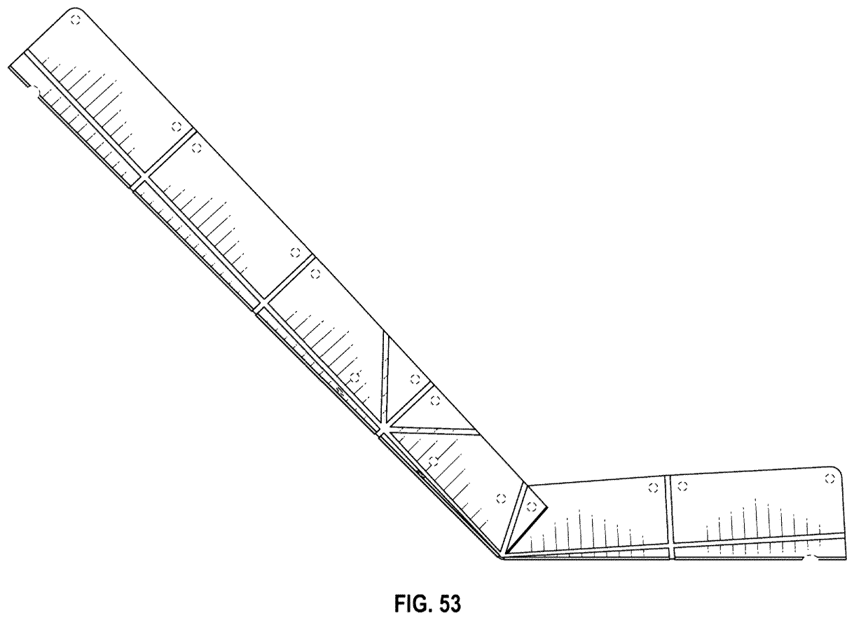

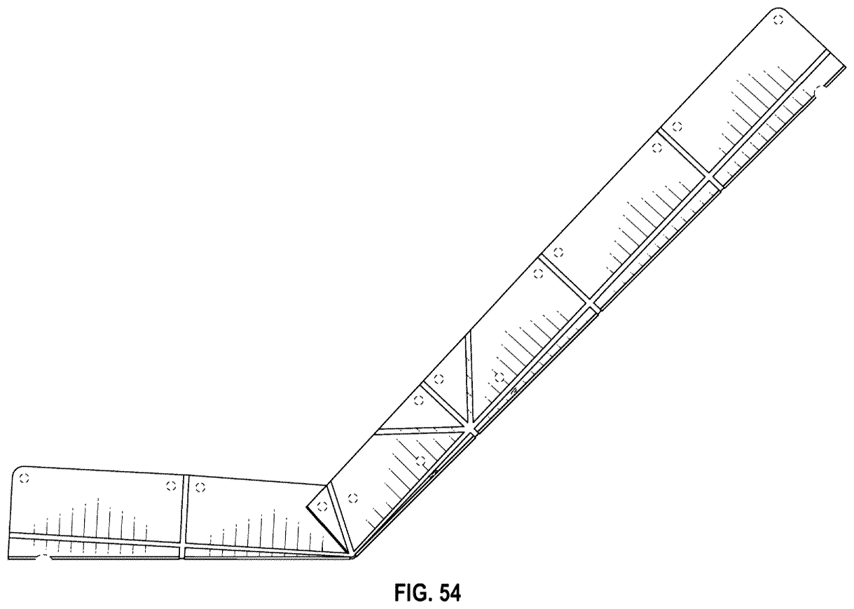

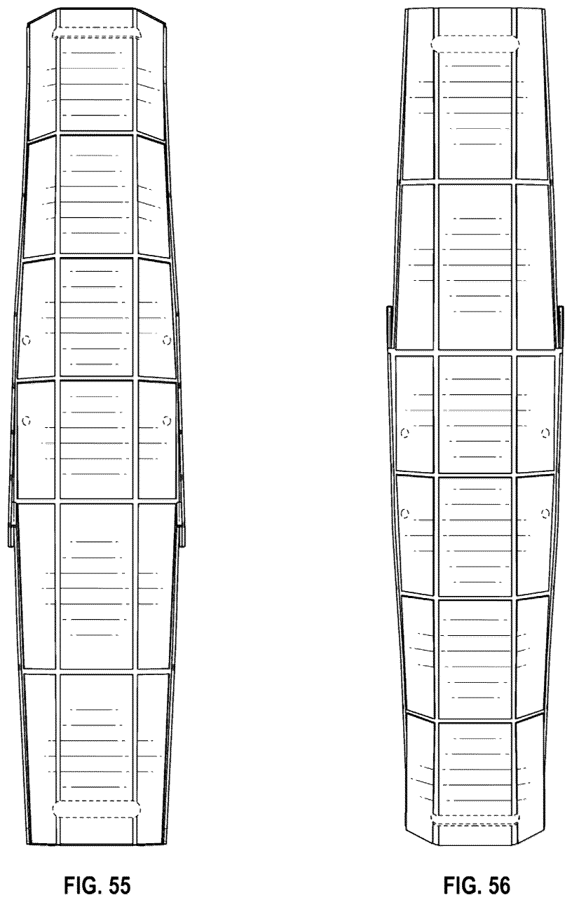

Referring to FIGS. 15-35 and 43-56, in some embodiments, the main body is folded into various deployed configurations. In each deployed configuration, the panel connection angle is at or between 0 and 180 degrees. In each deployed configuration, the outermost segments of each panel are folded at an angle between 0 and 180 degrees. Thus, the segment connection angle is between 0 and 180 degrees. This folding at the segment connection angle within a panel provides strength and rigidity to the device in the lengthwise direction when the device is in the deployed configuration.

In some embodiments, the main body defines one or more hole 106, such as a segment hole. In some embodiments, at least one segment of each panel includes a segment hole 106. In some embodiments, the holes are sized and shaped such that the segment holes align with one another when the main body is folded into the stowable configuration. In some embodiments, the holes are sized and shaped to receive a fastener 107, such as a strap, band, cord, nail, snap, or pin. The main body is maintained in the stowable configuration by threading the strap, band, cord, pin, or nail through the aligned holes. In some embodiments, the hole(s) 106 are elongated or slot-shaped. In some embodiments the strap, band, or cord is an elastic material. In some embodiments, the pin, snap, or nail is a rigid material, such as plastic or metal. In some embodiments, the strap is a zip tie or hook and loop fastener. In some embodiments, when the device is in the deployed configuration, the strap, band, cord, pin, or nail is threaded through the hole(s) to secure the device in the deployed configuration. In some embodiments, when the device is in the deployed configuration, the strap, band, cord, pin, or nail is threaded through the hole(s) to secure the device to the limb or extremity of a user or patient.

In some embodiments, the immobilization device is configured to be used with a tourniquet such that compressive loads are distributed across a larger area than is possible through the use of a tourniquet alone. Referring to FIG. 60, in some embodiments, when the device is in the deployed configuration, the strap, band, cord, pin, or nail is threaded through the hole(s) to secure the device body or trunk of the user, for example when used as a pelvic binder. In some embodiments, the present inventive concept is used in conjunction with a tourniquet such as described in U.S. Pat. Pub. No. US20150094756 as a pelvic binder. In some embodiments, the strap or band threaded through the holes (or slots) in the main body is a tourniquet such as described in U.S. Pat. Pub. No. US20150094756.

In some embodiments, the immobilization device in a deployed configuration is configured to restrain an appendage, limb, or extremity in a straight, 180 degree orientation. In some embodiments, the immobilization device in a deployed configuration is configured to restrain an appendage at an angle between 0 and 180 degrees, for example, 45 degrees or 90 degrees.

In some embodiments, the main body includes one or more pinch point 108. The pinch point is defined by opposed angle segments of adjacent segments of adjacent panels. The pinch point is configured to establish and maintain the panel connection angle at a predetermined angle when the opposed angle segments are folded over one another. In some embodiments, the pinch point includes one or more hole. The hole(s) are configured to receive a fastener. In this manner the opposed angle segments are fastened together when folded over one another. When the pinch point holes are aligned and a fastener is secured at the pinch point, the panels are secured in place establishing and maintaining the panel connection angle at the predetermined angle. In some embodiments, the fastener is a zip tie, nail, pin, brad, screw, or other fastener, such as a snap assembly. In some embodiments, each snap assembly includes a first component that is configured to engage with a corresponding second component, thereby securing the panels in the angled configuration. In some embodiments, the first and second components are coupled to the main body, thereby forming a main body assembly. In some embodiments, the main body defines clearance holes through which the first and/or second snap component extends when engaged with the corresponding other component.

In some embodiments, the present inventive concept relates to methods for using an immobilization device. The method includes moving the immobilization device from the stowable configuration to the deployed configuration. In some embodiments, the method also includes moving the immobilization device to flat configuration. In some embodiments, the method also includes placing the immobilization device in the deployed configuration on the patient's (or user's) extremity to immobilize the user's extremity. In some embodiments, the method also includes securing the immobilization device in the deployed configuration to the user via the strap, cord, tourniquet or other fastener. In some embodiments, the method further includes adjusting the size of the immobilization device to accommodate a specific need, such as by folding back excess material and/or by coupling two or more pieces of material together.

In some embodiments, the present inventive concept comprises a splint main body having a plurality of panels hingedly connected together, each panel having a plurality of segments hingedly connected together. In some embodiments, the main body is formed from a single piece of material defining a plurality of living hinges for defining and hingedly connecting the plurality of panels and segments. In some embodiments, the main body is formed from durable yet light-weight material, such as polypropylene, polyethylene, or any other light and durable material now known or later developed. In some embodiments, one or more panels of the plurality of panels is configured to fold over one or more other panels of the plurality of panels so as to optimize or otherwise improve the size of the device for a specific use.

The splint is moveable between a stowable configuration and a flat configuration. In the stowable configuration, the various panels are folded over onto one another so as to minimize the overall surface area of the splint, thereby increasing portability and stowability of the splint. In some embodiments, a fastener, such as cords, cables, straps, and/or one or more other elongated member, engages with one or more hole or slot of the splint, thereby retaining the splint in the stowed configuration. In some embodiments, the splint is included in a kit with one or more other medical device or supplies, such as gauze, gloves, or the like. In some embodiments, the one or more elongated member is formed from an elastic material, such as a bungee cord, elastic bands, or the like, thereby allowing the elongated member to selectively engage with and secure such other medical devices and/or supplies to and/or in relationship with the splint. In some embodiments, the splint and/or one or more elongated member includes a fastener to secure the splint to another object, such as to a belt, an article of clothing, a harness, a vehicle, or anywhere else it would be advantageous to have ready access to a splint or other medical supplies associated with the kit. In some embodiments, the fastener is a hook and loop fastener so that the splint and/or kit can be easily engaged with and/or disengaged from such other object.

In the flat configuration, each panel is parallel with, and extends from, each adjacent panel and each segment is parallel with, and extends from, each adjacent segment. From the flat configuration, the splint is selectively moveable to one of several deployed configurations. In a first deployed configuration the splint is configured to selectively restrain a person's arm or other appendage in a straight configuration such that the appendage is in an “open” (180) degree orientation. In a second deployed configuration the splint is configured to selectively restrain the appendage in an angled configuration. In some embodiments, the angle is 90 degrees. In other embodiments, the angle is between 180 and 90 degrees, such as 157 degrees (rotated 33 degrees away from the straight configuration), 135 degrees (rotated 45 degrees away from the straight configuration), or 123 degrees (rotated 67 degrees away from the straight configuration). In still other embodiments, the angle is less than 90 degrees.

In the first deployed configuration, each segment is rotated relative to an adjacent segment of the same panel while remaining generally parallel with each adjacent segment of adjacent panels. In this way, the splint gains rigidity for retaining each segment in general parallel relationship with each adjacent segment of adjacent panels. In the first deployed configuration, the splint is configured to wrap at least partially around a limb, such as an arm, and over a joint of the limb, such as the elbow, while the limb is in a straight configuration. In this way, the limb can be restrained in the straight configuration by securing the splint to the limb.

In the second deployed configuration, each segment is rotated relative to an adjacent segment of the same panel and at least some segments are rotated relative to an adjacent segment of an adjacent panel. In this way, the splint gains rigidity for retaining each segment in a general fixed relationship with each adjacent segment of adjacent panels. In the second deployed configuration, the splint is configured to wrap at least partially around a limb, such as an arm, and over a joint of the limb, such as the elbow, while the limb is in a bent configuration. In this way, the limb can be restrained in the bent configuration by securing the splint to the limb.

In some embodiments, the main body of the splint includes one or more pinch point for controlling the angle of the second deployed configuration. In some embodiments, the pinch point is created by forming opposed angled panels from opposed segments of one or more adjacent panel. In some embodiments, a first and second set of adjacent panels each includes respective opposed first and second outer segments and one or more respective inner segment extending therebetween. In some embodiments, each outer segment is divided into respective first 109 and second portions 110, with each first portion of each outer segment being hingedly coupled to each respective second portion while also being hingedly coupled to an adjacent first portion of an adjacent panel. In this way, the splint is moveable to the second deployed configuration by rotating respective first portions against each other, with the angle of the second deployed configuration being determined by the configuration of the first portions. In some embodiments, the first portions are configured to be secured together so as to secure the splint in the deployed configuration. In some embodiments, the first portions are configured to selectively rotate against and/or to be fastened to the second portion of at least one of the first and second panels, thereby providing greater strength and rigidity for retaining the angle of the second deployed configuration. In some embodiments, the main body of the splint includes a plurality of pinch points, thereby facilitating movement of the splint to a second deployed configurations, at one or more different angle.

In some embodiments, the main body of the splint defines one or more set of coordinating holes, such as circular holes, rectangular slots, or the like. In some embodiments, a first set of coordinating holes is configured such that a plurality of holes 106 through individual panels align when the splint is in the stowed configuration, creating a plurality of holes 106 through a plurality of panels so as to facilitate securing the splint in the stowed configuration. In some embodiments, a second set of coordinating holes is configured such that a plurality of holes 106 through individual segments can be utilized to engage with one or more strap, cord, or other elongated member for selectively securing the splint to a limb of a user.

In some embodiments, a third set of coordinating holes is configured such that a first hole 106 through a first portion of a first outer segment of a first panel is configured to align with a first hole 106 through a first portion of a first outer segment of a second panel, thereby enabling the respective first portions to be fastened together, such as with a bolt, a pin, a zip tie, or the like. In some embodiments, the second portion of the first outer segment of at least one of the first or second panels defines a second hole that is configured to align with the first holes when the first portions are folded against the respective second portion, thereby defining a stack-up hole through the stack-up of such portions so as to allow the first portions to be fastened to the respective second portion. In some embodiments, a third hole is defined by an adjacent inner segment such that a fastener, such as a zip tie, is able to extend from the third hole to the stack-up hole. In some embodiments, the fastener, such as a zip tie, extends through the stack-up hole and around an outer edge of the outer segment of the first or second panel.

In some embodiments, the splint is moveable from a flat configuration to a third deployed configuration. In the third deployed configuration, each panel is rotated relative to an adjacent panel and each segment is generally parallel with each adjacent segment of the same panel. In this way, the splint gains rigidity for retaining each segment in a general fixed relationship with each adjacent segment of the same panel. In the third deployed configuration, the splint is configured to wrap at least partially around a portion 150 of a person or animal, such as an abdomen or pelvis of such person or animal, thereby creating a barrier or support for the same. In some embodiments, the main body of the splint defines one or more engagement feature 120 (such as a slot) for engaging with a constriction assembly 125, such as a tourniquet of U.S. Pat. No. 9,855,055, the entire disclosure of which is incorporated herein by reference. In some embodiments, the engagement feature is configured to engage with (such as by receiving) an elongated member of the constriction assembly 125. In this way, the splint is capable of distributing compressive loads in a different manner (such as by distributing across a larger area) than is possible through the use of the constriction assembly alone. In some embodiments, the main body defines a plurality of engagement slots, thereby facilitating the adjustment of the device to fit a persons of various sizes and/or for accommodating various applications, such as by folding back excess material not required for a particular application. In some such embodiments, folding back excess material causes a first engagement slot to align with a second engagement slot, thereby allowing a tourniquet or other elongated member to simultaneously engage with each.

In use, the splint is moved from a stowed configuration to a flat configuration by unfolding respective panels away from each other. From the flat configuration, the splint can be moved to a variety of deployed configurations to satisfy a variety of needs. In some circumstances, panel segments and/or segment portions are folded relative to each other so as to create longitudinal and/or angular rigidity. In some circumstances, panels are folded relative to each other so as to form lateral rigidity. One or more cable, strap, tourniquet, or other elongated member can be used with the splint to secure the splint to a user and/or for generating compressive loads for spreading across a portion of the splint.

In some embodiments, the splint is designed so that it can be connected to one or more other splint, such as through one or more sets of coordinating holes, thereby creating splints of various sizes and/or shapes. In this way, the combined splints can provide rigidity for a limb or other portion of a body having a length or girth too large for one splint alone. In some embodiments, pelvic binding can be achieved by attaching two or more splints together, such as in respective third configurations, so that they cover the circumference of a portion of the pelvic girdle of a user, such as ¾ of the pelvic girdle. In some embodiments, the preferred girdle configuration enables a user to impose proper pressure on the pelvic girdle, such as by reducing improper pressure associated with pelvic girdles that are not properly fitted to the wearer. In some embodiments, the width of a single splint is configured to provide sufficient stabilization to bind the pelvic girdle for pelvic splinting. In some embodiments, the width of a single splint is seven (7) inches. In some embodiments, the splint is fabricated from a material that is form fitting and/or the splint is configured to otherwise create a snug, comfortable fit.

While the present general inventive concept has been shown in the drawings and fully described above with particularity and detail in connection with what is presently deemed to be the most practical and preferred embodiment(s) of the invention, it will be apparent to those of ordinary skill in the art that many modifications thereof may be made without departing from the principles and concepts set forth herein, including, but not limited to, variations in size, materials, shape, form, function and manner of operation, assembly and use.

It is also to be understood that the following claims are intended to cover all of the generic and specific features of the invention herein described, and all statements of the scope of the invention which, as a matter of language, might be said to fall there between. Hence, the proper scope of the present general inventive concept should be determined only by the broadest interpretation of the appended claims so as to encompass all such modifications as well as all relationships equivalent to those illustrated in the drawings and described in the specification.

Finally, it will be appreciated that the purpose of the annexed Abstract is to enable the U.S. Patent and Trademark Office and the public generally, and especially the scientists, engineers and practitioners in the art who are not familiar with patent or legal terms or phraseology, to determine quickly from a cursory inspection the nature and essence of the technical disclosure of the application. Accordingly, the Abstract is neither intended to define the invention or the application, which only is measured by the claims, nor is it intended to be limiting as to the scope of the invention in any way.

1. A binding device for localized binding of pelvic structures of a patient, the binding device comprising:
a first main body comprising a plurality of panels,
wherein said first main body is configured to encircle at least part of the pelvic structure,
wherein a first engagement feature of said first main body is configured to engage with an elongated member, and
wherein a first panel of said plurality of panels is configured to fold against a second panel of said plurality of panels.

2. The binding device of claim 1, wherein each panel of said plurality of panels is hingedly connected by a hinged connection to an adjacent panel of said plurality of panels.

3. The binding device of claim 2, wherein the hinged connection is a living hinge.

4. The binding device of claim 3, wherein said first engagement feature is an engagement slot defined by a proximal panel of said plurality of panels, said first engagement slot being configured to receive the elongated member.

5. The binding device of claim 4, wherein said first main body is configured to move between a flat configuration and a stowable configuration.

6. The binding device of claim 5, wherein each of said plurality of panels of said first main body is folded against at least one other panel of said plurality of panels when said first main body is in the stowable configuration.

7. The binding device of claim 1, wherein a proximal panel of a plurality of panels of said first main body is configured to fold against a second panel of said plurality of panels, thereby reducing the length of said first main body.

8. The binding device of claim 7, wherein said first engagement feature comprises a primary engagement slot defined by said primary panel, said primary engagement slot being configured to receive a portion of the elongated member.

9. The binding device of claim 8, wherein said second panel defines a secondary engagement slot that aligns with said primary engagement slot when said proximal panel is folded over said second panel.

10. The binding device of claim 1, further comprising a second main body coupled to said first main body, wherein:
the first engagement feature is a first engagement slot that is defined by a proximal panel of said first main body, said first engagement slot being configured to receive the elongated member; and
the second engagement feature is a second engagement slot that is defined by a distal panel of a plurality of panels of said second main body, said second engagement slot being configured to receive the elongated member.

11. A method for localized binding of pelvic structures of a patient, the method comprising:
encircling at least part of a pelvic structure with a first main body of a binding device;
engaging an elongated member with a first engagement feature of the first main body; and
constricting the elongated member.

12. The method of claim 11, wherein the first engagement feature is an engagement slot defined by a proximal panel of the first main body, and wherein engaging the elongated member with the engagement slot comprises extending the elongated member through the engagement slot.

13. The method of claim 12, further comprising moving the first main body from a stowable configuration to a flat configuration, the proximal panel of the first main body being folded against a second panel of the first main body when the first main body is in the stowable configuration.

14. The method of claim 13, further comprising engaging the elongated member with a second engagement feature, the second engagement feature being an engagement slot defined by a distal end of the first main body.

15. The method of claim 13, further comprising:
sizing the binding device to fit a user, wherein sizing the binding device comprises at least one of folding back excess portions of the first main body and coupling the first main body to a second main body; and
engaging the elongated member with a second engagement feature of the binding device.

16. A binding system for localized binding of pelvic structures of a patient, the binding system comprising:
an elongated member; and
a binding device comprising a first main body that is configured to encircle at least part of the pelvic structure, said binding device having a first engagement feature engaged with said elongated member.

17. The binding system of claim 16, wherein said first main body comprises a plurality of panels, each panel being hingedly connected to an adjacent panel of said plurality of panels, wherein a first panel of said plurality of panels of said first main body is configured to fold against a second panel of said plurality of panels of said first main body, thereby moving said first main body towards a stowable configuration.

18. The binding system of claim 16, wherein said first engagement feature comprises a primary engagement slot defined by a proximal panel of a plurality of panels of said first main body, and wherein said elongated member is received by said first engagement slot.

19. The binding system of claim 18, wherein said elongated member is engaged with a second engagement feature of said binding device, said second engagement feature comprising a primary engagement slot defined by a distal panel of one of said first main body and a second main body.

20. The binding system of claim 18, wherein said plurality of panels of said first main body comprises a second panel hingedly coupled to said proximal panel, said second panel defining a secondary engagement slot that aligns with said primary engagement slot of said proximal panel when said proximal panel is folded over onto said second panel such that said first engagement feature comprises said secondary engagement slot.
